(19) United States
(12) Patent Application Publication
(10) Pub. No.: US 2022/0188937 A1
(43) Pub. Date: Jun. 16, 2022

(54) DYNAMIC IDENTIFICATION OF COLLATERAL INFORMATION

(71) Applicant: International Business Machines Corporation, Armonk, NY (US)

(72) Inventors: Joao H. Bettencourt-Silva, Dublin (IE); Eoin Carroll, Dublin (IE); Vanessa Lopez Garcia, Dublin (IE); Marco Luca Sbodio, Dublin (IE)

(21) Appl. No.: 17/121,928

(22) Filed: Dec. 15, 2020

(51) Int. Cl. G06Q 40/08; G06Q 10/10; G06Q 30/00; G16H 50/20; G16H 40/20; G16H 10/60; G06N 20/00; G06F 16/903

(52) U.S. Cl. CPC G06Q 40/08; G06Q 10/10; G06Q 30/0185; G16H 50/20; G06Q 50/26; G16H 10/60; G06N 20/00; G06F 16/90335; G16H 40/20

(57) ABSTRACT

Embodiments of the present invention provide a computer system, a computer program product, and a method that comprises identifying claim data from a received data set associated with a user; analyzing the identified claim data based on a historical database of claim information associated with the user; retrieving expert information associated with the analysis of the identified claim data; extracting the identified claim data based on a comparison of the analysis of identified claim data and the retrieved expert information associated with the claim data by normalizing a range of features of the analyzed data; dynamically determining an overall risk score associated with the extracted claim data; and dynamically transmitting the extracted claim data to a different computing device associated with a different user.

### BACKGROUND

The present invention relates generally to the field of healthcare technology, and more specifically regulation associated with compliance and policy of insurance claims.

Compliance means conforming to a rule, such as a specification, policy, standard or law. Regulatory compliance describes the goal that organizations aspire to achieve in their efforts to ensure that they are aware of and take steps to comply with relevant laws, policies, and regulations. Due to the increasing number of regulations and need for operational transparency, organizations are increasingly adopting the use of consolidated and harmonized sets of compliance controls. This approach is used to ensure that all necessary governance requirements can be met without the unnecessary duplication of effort and activity from resources. Some organization keep compliance data—all data belonging or pertaining to the enterprise or included in the law, which can be used for the purpose of implementing or validating compliance—in a separate store for meeting reporting requirements. Compliance software is increasingly being implemented to help companies manage their compliance data more efficiently.

A compliance management system (“CMS”) consists of an integrated system of written documents, processes, tools, controls, and functions to make it easier for organizations to comply with legal requirements. A CMS also minimizes harm to consumers because of a violation of law. A CMS helps organizations better address risk management by ensuring that their policies and procedures adhere to the requirements of applicable laws and regulations, as well as address training, communication, and monitoring.

### SUMMARY

Embodiments of the present invention provide a computer system, a computer program product, and a method that comprises identifying claim data from a received data set associated with a user; analyzing the identified claim data based on a historical database of claim information associated with the user; retrieving expert information associated with the analysis of the identified claim data; extracting the identified claim data based on a comparison of the analysis of identified claim data and the retrieved expert information associated with the claim data by normalizing a range of features of the analyzed data; dynamically determining an overall risk score associated with the extracted claim data; and dynamically transmitting the extracted claim data to a different computing device associated with a different user.

### BRIEF DESCRIPTION OF THE DRAWINGS

FIG. 1 is a functional block diagram depicting an environment with a computing device connected to or in communication with another computing device, in accordance with at least one embodiment of the present invention;

FIG. 2 is a flowchart illustrating operational steps for identifying compliance requirements attached to an insurance claim associated with a respective user, in accordance with at least one embodiment of the present invention;

FIG. 3 is a flowchart illustrating operational steps for communicating required collateral information to providers during a submission process, in accordance with at least one embodiment of the present invention;

FIG. 4 is an exemplary diagram depicting an identification of compliance requirements attached to an insurance claim associated with a respective user, in accordance with at least one embodiment of the present invention; and

FIG. 5 depicts a block diagram of components of computing systems within a computing display environment of FIG. 1, in accordance with an embodiment of the present invention.

### DETAILED DESCRIPTION

Embodiments of the present invention recognize the need for an improvement to insurance claim issue response technology systems due to an amount of resources needed to identify reimbursement and eligibility requirements for each insurance claim filed by a provider associated with each respective user. Current insurance claim issue response technology identity reimbursement and eligibility requires by performing separate queries for each of the following: eligible providers, eligible places of service, prior authorization requirements, documentation of medical necessity and other limitation of service that the provider associated with each user in a plurality of users in a given period. The common given period is commonly the post-payment period. Generally, insurance claim issue response technology is pure post-payment fraud, waste, and abuse detection, which requires labor-intensive and often abrasive solutions. Embodiments of the present invention improve the efficiency and cost by reducing delays in payment and reducing the intensity in labor required for current insurance claim issue response technology systems by comparing identified claim data to policy rules and matching eligibility data during a pre-payment process, which is a period prior to the common post-payment period. Embodiments of the present invention reduce the cost of delays of payment and improves efficiency of current insurance claim issue response technology by comparing identified claim data to a plurality of policy rules and reduces the intensity in labor by matching eligibility factors associated with the identified claims based on a dynamically determined overall risk score associated with the identified eligibility factors meeting or exceeding a predetermined threshold of risk within a given period. Embodiments of the present invention identify eligibility factors associated within the identified claim data, which reduces the intensity in labor required for current insurance claim issue response technology systems to perform individual quires for the identified claim, by analyzing claim data based on a historical database of claim data associated with a user; retrieving expert information associated with the claim data; scaling an extraction of claim data based on a comparison of the historical database of claim data and retrieved expert information associated with the claim data; dynamically determining an overall risk score associated with the claim data based on a summation assigned weighted values for a plurality of factors associated with the claim data; and performing a query for additional data associated with the user in response to the overall risk score meeting or exceeding a predetermined threshold of risk. In this embodiment, the program 104 defines the additional data associated with the user as collateral information and information required by a payer to be attached with the claims data that has an impact on claim denial and reimbursement issues. In this embodiment, the program defines collateral information as information that provides evidence and increase the insurance of billing compliance associated with the claim data. For example, collateral information includes medical records, medical notes, and social care notes. In this embodiment, the program defines the billing compliance of the claim data as a condition associated with the claim data that ensures that the completeness of required information and validity of the information with respect to a plurality of eligibility compliance requirements. In this embodiment, the program defines a plurality of compliance requirements as a set of pre-conditions (formally or informally expressed) that must true to ensure that the user is eligible for the claim a cost of service.

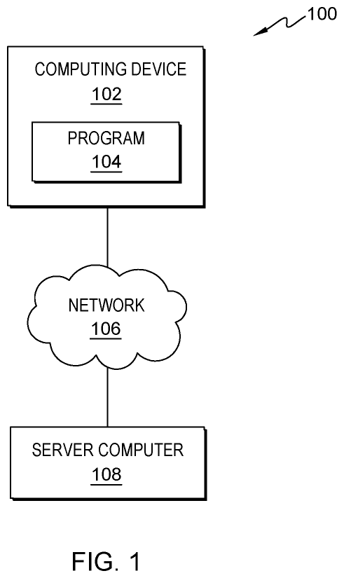

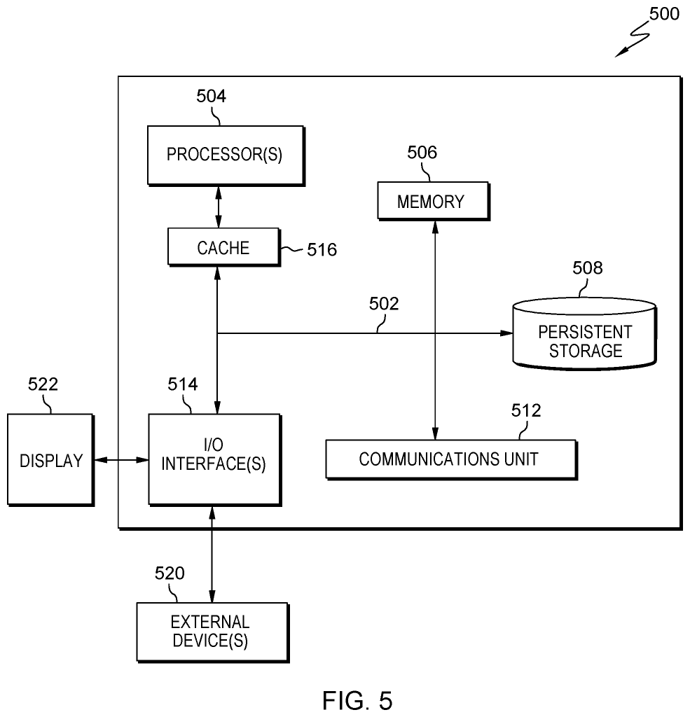

FIG. 1 is a functional block diagram of a computing environment 100 in accordance with an embodiment of the present invention. The computing environment 100 includes a computing device 102 and a server computer 108. The computing device 102 and the server computer 108 may be desktop computers, laptop computers, specialized computer servers, smart phones, wearable technology, or any other computing devices known in the art. In certain embodiments, the computing device 102 and the server computer 108 may represent computing devices utilizing multiple computers or components to act as a single pool of seamless resources when accessed through a network 106. Generally, the computing device 102 and the server computer 108 may be representative of any electronic devices, or a combination of electronic devices, capable of executing machine-readable program instructions, as described in greater detail with regard to FIG. 5. In this embodiment, the computing device 102 may be a computing device 102 associated with a bank, service provider, dentist, hospital, or corporation.

The computing device 102 may include a program 104. The program 104 may be a stand-alone program on the computing device 102. In another embodiment, the program 104 may be stored on a server computer 108. In this embodiment, the program 104 reduces delays in payment of insurance claims and reduces the intensity in labor required for current insurance claim issue response technology systems by comparing identified claim data to policy rules and matching eligibility data during a pre-payment process, which is a period prior to the common post-payment period. In this embodiment, the program 104 compares identified claim data to policy rules and matches eligibility data during this earlier period of fixed time using a feedback collector component (not shown), which improves the efficiency and reduces the cost associated with current insurance claim issue response technologies, by dynamically determining an overall risk score associated with the identified claim data based on a summation of assigned weight values for each identified eligibility factor meeting or exceeding a predetermined threshold of risk within a given period of time. In this embodiment, the program 104 dynamically determines the overall risk score associated with the identified claim data by identifying claim data associated with a user; analyzing the identified claim data based on a historical database of claim data associated with a user; retrieving expert information associated with the claim data; scaling an extraction of claim data based on a comparison of the historical database of claim data and retrieved expert information associated with the claim data; dynamically determining an overall risk score associated with the claim data based on a summation assigned weighted values for a plurality of factors associated with the claim data; and performing a query for additional data associated with the user in response to the overall risk score meeting or exceeding a predetermined threshold of risk.

In this embodiment, the program 104 identifies claim data from a larger sample of received data associated with the user by determining claim data from the remainder of received data using a pattern recognition algorithm. For example, the program 104 identifies the services provided to the user, the amount covered by the insurance of the user, and the credit score associated with the user. In this embodiment, the program 104 defines claim data as data that provides information associated with a service provided attached to an insurance claim associated with the user. For example, the program 104 identifies a substantive diagnosis and a cost associated with the diagnosis as identified claim data. Then, the program 104 analyzes the identified claim data by identifying a plurality of eligibility factors using a machine learning algorithm and an artificial intelligence algorithm based on a historical database associated with the user. For example, the program 104 identifies the age of the user, the current insurance provider of the user, and the insurance primum associated with the user as eligibility factors. Then, the program 104 retrieves expert information associated with the analysis of the identified claim data by performing a query of expert opinions within an opinion database. For example, the program 104 retrieves eligibility requirements associated with a provided service, and these eligibility requirements are policies based on expert information. In this embodiment, the program 104 extracts the plurality of eligibility factors of the identified claim data based on a comparison of the identified claim data to the retrieved expert information and the analysis of the identified claim data. In this embodiment, the program 104 transmits instructions to an eligibility compliance requirement extractor (not shown) to extract the plurality of eligibility factors of the identified claim data. In this embodiment, the program 104 defines the eligibility factors as indicative markers that determine whether the claim data associated with the user complies with the retrieved expert information. For example, the program 104 extracts and separates the policy information associated with the service provided to the user and the insurance plan associated with the user from the remainder of the claim data. In this embodiment, the program 104 defines extraction as the process of retrieving data of data sources for further data processing. In this embodiment, the program 104 compares the identified claim data to the retrieved expert information and the analysis of the identified claim data by matching factors associated with the identified claim data. In another embodiment, the program 104 extracts collateral information also from the identified claim data. In this embodiment, the program 104 transmits instructions to a collateral information extractor (not shown) to extract and separate the collateral information from the identified claim data. In this embodiment, the program 104 dynamically determines the overall risk score associated with the extracted identified claim data by summing assigned weight values for each factor associated with the claim data for the user. In this embodiment, the program 104 defines the determined overall risk score as an explanation. In this embodiment, the program 104 defines the explanation as the degree of risk of defaulting a payment associated with the claim data. For example, the program 104 determines the overall risk score of a user is 4 based on the summing of the insurance plan associated with the user having an assigned weighted value of 2, the service provided to the user having an assigned value of 1, and the collateral information associated with the user having an assigned value of 1. In this embodiment and in response to the determined overall risk score meeting or exceeding the predetermined threshold of risk, dynamically transmitting obtained additional information associated with the claim data to another computing device 102 associated with a different user by performing a query for the identified eligibility factors and the collateral information associated with the user. In this embodiment, the program 104 defines the predetermined threshold of risk as a level of risk associated with the explanation that indicates that an insurance claim may be defaulted. In this embodiment and in response to the overall risk score failing to meet the predetermined threshold of risk, the program 104 will not obtain additional information associated with the identified claim data. For example, the predetermined threshold of risk associated with the service provided is 4, and the program 104 determined the calculated overall risk score associated with the identified claim data of the user is 4. In this example, the calculated overall risks score meets the predetermined risk, therefore the program 104 retrieves billing information, billing history associated with the user, and a credit score associated with the user prior to the payment period. In this embodiment, the program 104 transmits instructions to a reporting component (not shown) associated with the computing device 102 to transmit information to another computing device associated with another user. In this embodiment, the program 104 defines additional details as information that has an impact on an ability of a payer processing a claim and has an impact on the calculation of the overall risk score.

The network 106 can be a local area network (“LAN”), a wide area network (“WAN”) such as the Internet, or a combination of the two; and it may include wired, wireless or fiber optic connections. Generally, the network 106 can be any combination of connections and protocols that will support communication between the computing device 102 and the server computer 108, specifically the program 104 in accordance with a desired embodiment of the invention.

The server computer 108 communicates with the computing device 102 via the network 106. In this embodiment, the program 104 transmits the extracted claim data to the server computer 108 for storage via the network 106. In another embodiment, the program 104 may be stored on the server computer 108. The server computer 108 may be a single computing device, a laptop, a cloud-based collection of computing devices, a collection of servers, and other known computing devices. In this embodiment, the server computer 108 may be in communication with the computing device 102. In another embodiment, the server computer 108 may be communication with the program 104. In another embodiment, the program 104 may store any insurance data, eligibility requirements, collateral information, and an evaluation on the server computer 108. In another embodiment, the reporting component may be located on the server computer 108 that receives transmitted instructions from the program 104 to transmit information to another server computing device.

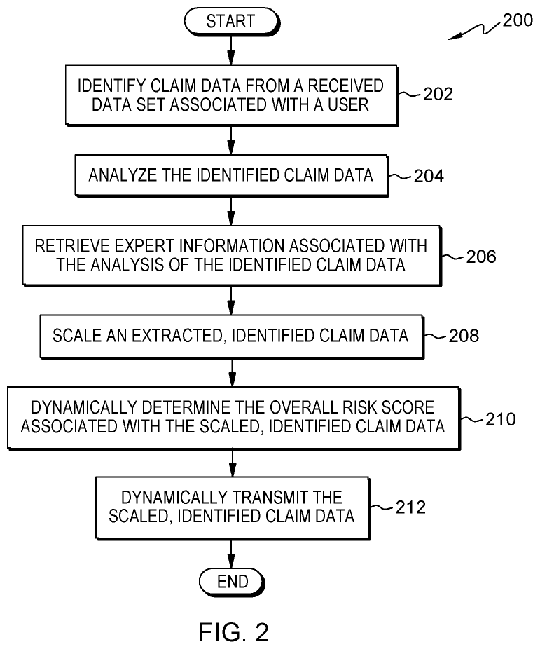

FIG. 2 is a flowchart 200 illustrating operational steps for identifying compliance requirements attached to an insurance claim associated with a respective user, in accordance with at least one embodiment of the present invention.

In step 202, the program 104 identifies claim data from a received data set associated with a user. In this embodiment, the program 104 identifies claim data from the received data set by determining claim data from the received data set using a pattern recognition algorithm. In this embodiment, the program 104 receives the data set associated with the user and contains more information than needed for processing a claim for services provided. For example, the program 104 identifies clinical data, policy and benefit manuals, and personal information associated with the user. In this embodiment, the program 104 receives opt-in/opt-out permission for access to the received data set from the user. In another embodiment, the program 104 generates and transmits a notification to the user in response to the program 104 accessing the received data set.

In step 204, the program 104 analyzes the identified claim data. In this embodiment, the program 104 analyzes the identified claim data based on a historical database of claim information associated with the user using machine learning algorithms and artificial intelligence algorithms. In this embodiment, the program 104 learns claim data that has an impact on an explanation of the identified claim data based on the analysis of the identified claim data and a comparison of the identified claim data to the historical database of claim information associated with the user. In this embodiment, the program 104 defines the explanation as a calculated overall risk score. For example, the program 104 compares eligibility information, prior authorization requirements, and documentation of medical necessity within the identified claim data from the received data set to the eligibility information, prior authorization requirements, and documentation of medical necessity from a historical database associated with the user. In this example, the program 104 detects a change in the eligibility information based on the analysis of the comparison of the identified claim data to the data stored in the historical database associated with the user. In this embodiment, the program 104 analyzes the identified claim data by identifying a plurality of eligibility factors using a machine learning algorithm and an artificial intelligence algorithm based on the historical database of information associated with the user.

In step 206, the program 104 retrieves expert information associated with the analysis of the identified claim data. In this embodiment, the program 104 retrieves expert information associated with the analysis of the identified claim data by performing a query of expert opinions associated with the identified claim data. In this embodiment, the program 104 defines an expert as an individual who has a comprehensive and authoritative knowledge of or skill in a particular matter. In this embodiment, the program 104 compares the identified eligibility factors associated with the analysis of the identified claim data to eligibility factors associated with a plurality of expert opinions by matching the eligibility factors based on compliance requirements. For example, the program 104 compares the insurance plan and provided service associated with the user to the type of insurance plan accepted at the service provider, the extent of coverage associated with the insurance plan of the user, and estimated out-of-pocket cost associated with the insurance plan of the user for the service provided. In this embodiment, the program 104 matches the plurality of eligibility factors by linking commonalities identified within the plurality of eligibility factors based on a predetermined set of pre-processing conditions associated with the claim data. For example, the program 104 retrieves expert information associated with the ailment of the service rendered to learn the general price and associated risks with the service provided, where the general price and associated risks are linked commonalities within the expert information.

In step 208, the program 104 extracts identified claim data. In this embodiment, the program 104 scales extracted, identified claim data based on the comparison of the analysis of identified claim data and the retrieved expert information associated with the claim data by normalizing a range of features of data. In this embodiment, the program 104 defines scaling as a process of standardizing the data ensuring uniformity throughout the data. In this embodiment, the program 104 scales the identified claim data by assigning weighted values to each identified factor. In this embodiment, the program 104 extracts identified claim data by importing the identified claim data into an intermediate database prior to the normalizing of the received data, which makes each factor uniformly scaled for summation. In another embodiment, the program 104 adds metadata (e.g., collateral information) to the identified claim data during the importation of the identified claim data into the intermediate database. For example, the program 104 adds the collateral information to the identified claim data associated with the user.

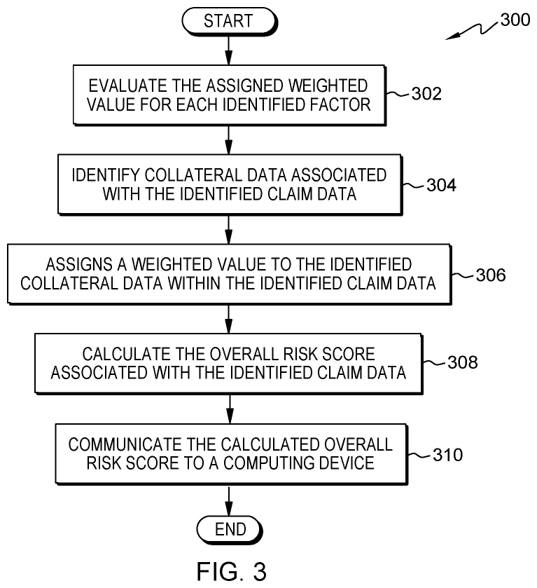

In step 210, the program 104 dynamically determines the overall risk score associated with the extracted, identified claim data. In this embodiment, the program 104 dynamically determines the overall risk score associated with the scaled, identified claim data based on a summation of the assigned weighted values for each identified factor. In this embodiment, the program 104 dynamically determines the overall risk score associated with the scaled identified claim by evaluating the assigned weighted values for each identified factor associated with the claim data; identifying collateral information associated with the identified claim data; assigning a weighted value to the identified collateral information associated with the identified claim data; calculating an overall risk score by summing the assigned weight values for each identified factor and the identified collateral information; and communicating with a service provider in response to the calculated overall risk score meeting or exceeding a predetermined threshold of risk. This step will be further explained in FIG. 3. In this embodiment, the program 104 evaluates the assigned weighted values for each identified factor by determining the identified factors that are usable for calculating risk estimates associated with the scaled, identified claim data. In this embodiment, the program 104 identifies collateral information by accessing a collateral information database associated with the user and selecting collateral information that has an impact on the calculated overall risk score. In this embodiment, the program 104 assigns a weighted value to the identified collateral information by scaling the information using a normalization algorithm. In this embodiment, the program 104 communicates with a service provider by establishing a line of communication using the network 106 and transmitting a risk level associated with the calculated overall risk score. In this embodiment, the program 104 generates a range of 1-3, with 3 being the highest risk level and 1 being the lowest risk level associated with the claim data.

In step 212, the program 104 dynamically transmits the extracted, identified claim data to a different computing device associated with a different user. In this embodiment, the program 104 transmits instructions to the reporting component to transmit the extracted, identified claim data to another computing device 102 associated with a different user in a plurality of users via the network 106. In this embodiment and in response to the determined overall risk score meeting or exceeding the predetermined threshold of risk, the program 104 dynamically transmits the extracted, identified claim data to the computing device 102 associated with the service provider via the network 106. In another embodiment, the program 104 dynamically transmits the scaled, identified claim data to a server computer 108 via the network in response to the determined overall risk score meeting or exceeding the predetermined threshold of risk.

FIG. 3 is a flowchart 300 illustrating operational steps for dynamically determines the overall risk score associated with the extracted, identified claim data, in accordance with at least one embodiment of the present invention.

In step 302, the program 104 evaluates the assigned weighted values for each identified factor associated with the identified claim data. In this embodiment, the program 104 evaluates the assigned weighted values for each identified factor associated with the identified claim data by analyzing the performance of the normalization algorithm used by the program 104 to standardize the identified claim data. In this embodiment, the program 104 analyzes the evaluated assigned weight values for each identified factor by comparing the each identified factor associated with the identified claim data to the eligibility requirements retrieved from the performed query of the expert information. In this embodiment, the program 104 evaluates the assigned weighted values by determining whether the identified claim data is usable for calculating risk estimates associated with the identified claim data. In this embodiment, the program determines whether the identified claim data is usable based on a positive match between each respective identified factor to each eligibility requirement associated with the expert information. For example, the program 104 evaluates the service provided, an identified factor, as an assigned weight of 2 based on a determination that the service provided is within a range of the eligibility requirements that is a risk.

In step 304, the program 104 identifies collateral data associated the identified claim data. In this embodiment and in response to evaluating the assigned weight values of each identified factor associated with the identified claim data, the program 104 identifies collateral data within associated with the user by accessing information databases associated with the user, and identifying additional information that has an impact on the determination of the overall risk score associated with the identified claim data. For example, the program 104 identifies pre-existing condition eligibility requirements within a medical records database associated with an insurance provider. In this embodiment, the program 04 access information databases by transmitting instructions to the information database that stores the collateral information to allow for access. In this embodiment, the program 104 determines whether the additional information has an impact on the overall risk score by evaluating the additional information using the retrieved expert information. For example, the program 104 identifies the medical records associated with the user as collateral data by accessing a health insurance database and evaluating the impact of the medical records on the overall risk score associated with the identified claim data. In another embodiment, the program 104 identifies an absence of collateral data in response to being denied access to a database that stores collateral data associated with the identified claim data.

In step 306, the program 104 assigns a weighted value to the identified collateral data within the identified claim data. In this embodiment, the program 104 assigns the weighted values to the identified collateral data within the identified claim data by scaling the identified collateral information using the normalization algorithm that was used on the identified claim data. In this embodiment, the program 104 uniformly applies these weighted values to the identified claim data and the identified collateral data; and based on this uniform application of assigned weighted values, the data is subject to mathematical manipulation. For example, the program 104 assigns a weighted value of 3 to the collateral information, which is the highest level of risk associated with the identified claim data, based on the pre-existing conditions present within the medical records associated with the user.

In step 308, the program 104 calculates the overall risk score associated with the identified claim data. In this embodiment, the program 104 calculates the overall risk score associated with the identified claim data by summing the assigned weighted values for each respective identified factor associated with the identified claim data and the assigned weight value for the identified collateral information associated with the user.

In step 310, the program 104 communicates the calculated overall risk score to a computing device 102. In this embodiment and in response to the calculated overall risk score meeting or exceeding a predetermined threshold of risk, the program 104 establishes a line of communication with the computing device 102 associated with the service provider and transmits the identified claim data and identified collateral information based on the calculated overall risk score. In this embodiment, the program 104 establishes the line of communication between a plurality of computing devices 102 via the network 106. In another embodiment, the program 104 establishes a line of communication between the computing device 102 and a server computer 108 via the network 106.

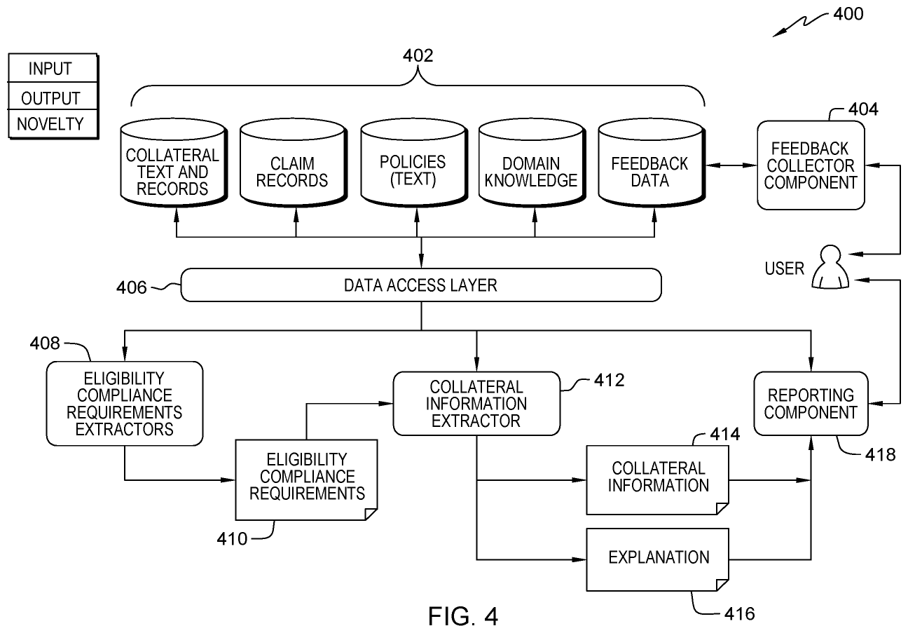

FIG. 4 is an exemplary diagram 400 depicting an identification of compliance requirements attached to an insurance claim associated with a respective user, in accordance with at least one embodiment of the present invention.

In exemplary diagram 400, data input 402 comprises collateral text and records, claim records, policies associated with claims, expert domain knowledge, and feedback data. The data input 402 flows into a feedback collector component 404 and a data access layer 406. The data access layer 406 provides simplified access to data stored in a persistent storage of some kind, such as an entity-relational database. The feedback collector component 404 collects feedback associated with the claim data from the user. The program 104 analyzes the data input 402 stored within the data access layer 406 using machine learning algorithms and artificial intelligence algorithms. In this embodiment, the program 104 identifies claim data within the data input 402 stored within the data access layer 406 based on the analysis of the data input 402. The program 104 extracts a plurality of eligibility factors from the identified claim data within the data input 402 using an eligibility compliance requirement extractor 408. In this embodiment, the program 104 compares the extracted plurality of eligibility factors to a plurality of eligibility compliance requirements 410. The program 104 assigns weighted values for each respective eligibility factors within the plurality of eligibility factors using the eligibility compliance requirement extractor 408. In this embodiment, the program 104 standardizes the data input 402 within the eligibility compliance requirement extractor 408 by assigning weighted values for each respective eligibility factor stored within the data access layer 406. The program 104 identifies collateral information 414 within the data access layer 406 and extracts the identified collateral information 414 from the data input 402 using a collateral information extractor 412. The collateral information extractor 412 analyzes the data input 402 within the data access layer 406; identifies collateral informational 414 within the data input 402; and assigns a weighted value for the identified collateral information 414 using machine learning algorithms and artificial intelligence algorithms. In this embodiment, the program 104 calculates an overall risk score by summing the assigned weight values for the extracted eligibility factors associated with the plurality of eligibility compliance requirements 410 and the assigned weighted value for the identified collateral information extractor 412. In this embodiment, the program 104 depicts the calculated overall risk score as an explanation 416. The explanation 416 is the calculated overall risk score associated with the data input 402 stored within the data access layer 406. In response to the explanation 416 meeting or exceeding a predetermined threshold of risk, the program 104 establishes a line of communication via the network 106 and transmits the data input 402 to a reporting component 418. The reporting component 418 is located within a computing device 102 associated with a service provider. In another embodiment, the reporting component 418 may be a server computer 108. The user may access the reporting component 418 based on a level of risk associated with the user and the data input 402 associated with the user.

FIG. 5 depicts a block diagram of components of computing systems within a computing environment 100 of FIG. 1, in accordance with an embodiment of the present invention. It should be appreciated that FIG. 5 provides only an illustration of one implementation and does not imply any limitations with regard to the environments in which different embodiments can be implemented. Many modifications to the depicted environment can be made.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

A computer system 500 includes a communications fabric 502, which provides communications between a cache 516, a memory 506, a persistent storage 508, a communications unit 512, and an input/output (I/O) interface(s) 514. The communications fabric 502 can be implemented with any architecture designed for passing data and/or control information between processors (such as microprocessors, communications and network processors, etc.), system memory, peripheral devices, and any other hardware components within a system. For example, the communications fabric 502 can be implemented with one or more buses or a crossbar switch.

The memory 506 and the persistent storage 508 are computer readable storage media. In this embodiment, the memory 506 includes random access memory (RAM). In general, the memory 506 can include any suitable volatile or non-volatile computer readable storage media. The cache 516 is a fast memory that enhances the performance of the computer processor(s) 504 by holding recently accessed data, and data near accessed data, from the memory 506.

The program 104 may be stored in the persistent storage 508 and in the memory 506 for execution by one or more of the respective computer processors 504 via the cache 516. In an embodiment, the persistent storage 508 includes a magnetic hard disk drive. Alternatively, or in addition to a magnetic hard disk drive, the persistent storage 508 can include a solid state hard drive, a semiconductor storage device, read-only memory (ROM), erasable programmable read-only memory (EPROM), flash memory, or any other computer readable storage media that is capable of storing program instructions or digital information.

The media used by the persistent storage 508 may also be removable. For example, a removable hard drive may be used for the persistent storage 508. Other examples include optical and magnetic disks, thumb drives, and smart cards that are inserted into a drive for transfer onto another computer readable storage medium that is also part of the persistent storage 508.

The communications unit 512, in these examples, provides for communications with other data processing systems or devices. In these examples, the communications unit 512 includes one or more network interface cards. The communications unit 512 may provide communications through the use of either or both physical and wireless communications links. The program 104 may be downloaded to the persistent storage 508 through the communications unit 512.

The I/O interface(s) 514 allows for input and output of data with other devices that may be connected to a mobile device, an approval device, and/or the server computer 108. For example, the I/O interface 514 may provide a connection to external devices 518 such as a keyboard, keypad, a touch screen, and/or some other suitable input device. External devices 518 can also include portable computer readable storage media such as, for example, thumb drives, portable optical or magnetic disks, and memory cards. Software and data used to practice embodiments of the present invention, e.g., the program 104, can be stored on such portable computer readable storage media and can be loaded onto the persistent storage 508 via the I/O interface(s) 514. The I/O interface(s) 514 also connect to a display 522.

The display 522 provides a mechanism to display data to a user and may be, for example, a computer monitor.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
identifying claim data from a received data set associated with a user;
analyzing the identified claim data based on a historical database of claim information associated with the user;
retrieving expert information associated with the analysis of the identified claim data;
extracting the identified claim data based on a comparison of the analysis of identified claim data and the retrieved expert information associated with the claim data by normalizing a range of features of the analyzed data;
dynamically determining an overall risk score associated with the extracted claim data; and
in response to the determined overall risk score meeting or exceeding a predetermined threshold of risk, dynamically transmitting the extracted claim data to a different computing device associated with a different user.

2. The computer-implemented method of claim 1, wherein analyzing the identified claim data based on the historical database of claim information comprises:
determining the claim data within the identified data has a quantitative impact on an overall risk score of the identified claim data;
identifying a plurality of eligibility factors within the identified claim data based on the historical database of information associated with the user using a machine learning algorithm and an artificial intelligence algorithm; and
comparing the determined claim data to the identified plurality of eligibility factors based on the historical database of claim information associated with the user.

3. The computer-implemented method of claim 1, wherein retrieving expert information comprises:
performing a query for expert information associated with the analyzed claim data;
matching at least two eligibility factors within a plurality of eligibility factors associated with each expert information within the query of expert opinions based on compliance requirements; and
retrieving the expert information with at least two matching eligibility factors within the plurality of eligibility factors.

4. The computer-implemented method of claim 3, wherein matching the at least two eligibility factors comprise linking commonalities within the plurality of eligibility factors based on a predetermined set of pre-processing conditions associated with the analyzed claim data.

5. The computer-implemented method of claim 1, wherein extracting the identified claim data comprises importing the identified claim data into an intermediate database prior to the normalizing of the received data.

6. The computer-implemented method of claim 5, further comprising adding metadata to the extracted claim data during the importation of the identified claim data into the intermediate database, wherein the metadata is collateral information associated with the user.

7. The computer-implemented method of claim 1, wherein dynamically determining the overall risk score associated with the extracted claim data comprises:
identifying a plurality of factors associated with collateral information associated with the extracted claim data;
assigning a weighted value to each identified factor within the identified plurality of factors based on the collateral information associated with the extracted claim data; and
calculating an overall risk score by summing the assigned weight values for each identified factor based on the collateral information associated with the extracted claim data.

8. A computer program product comprising:
one or more computer readable storage media and program instructions stored on the one or more computer readable storage media, the program instructions comprising:
program instructions to identify claim data from a received data set associated with a user;
program instructions to analyze the identified claim data based on a historical database of claim information associated with the user;
program instructions to retrieve expert information associated with the analysis of the identified claim data;
program instructions to extract the identified claim data based on a comparison of the analysis of identified claim data and the retrieved expert information associated with the claim data by normalizing a range of features of the analyzed data;
program instructions to dynamically determine an overall risk score associated with the extracted claim data; and
in response to the determined overall risk score meeting or exceeding a predetermined threshold of risk, program instructions to dynamically transmit the extracted claim data to a different computing device associated with a different user.

9. The computer program product of claim 8, wherein the program instructions to analyze the identified claim data based on the historical database of claim information comprise:
program instructions to determine the claim data within the identified data has a quantitative impact on an overall risk score of the identified claim data;
program instructions to identify a plurality of eligibility factors within the identified claim data based on the historical database of information associated with the user using a machine learning algorithm and an artificial intelligence algorithm; and
program instructions to compare the determined claim data to the identified plurality of eligibility factors based on the historical database of claim information associated with the user.

10. The computer program product of claim 8, wherein the program instructions to retrieve expert information comprise:
program instructions to perform a query for expert information associated with the analyzed claim data;
program instructions to match at least two eligibility factors within a plurality of eligibility factors associated with each expert information within the query of expert opinions based on compliance requirements; and
program instructions to retrieve the expert information with at least two matching eligibility factors within the plurality of eligibility factors.

11. The computer program product of claim 10, wherein the program instructions to match the at least two eligibility factors comprise program instructions to link commonalities within the plurality of eligibility factors based on a predetermined set of pre-processing conditions associated with the analyzed claim data.

12. The computer program product of claim 8, wherein the program instructions to extract the identified claim data comprise program instructions to import the identified claim data into an intermediate database prior to the normalizing of the received data.

13. The computer program product of claim 12, wherein the program instructions stored on the one or more computer readable storage media further comprise:
program instructions to add metadata to the extracted claim data during the importation of the identified claim data into the intermediate database, wherein the metadata is collateral information associated with the user.

14. The computer program product of claim 8, wherein the program instructions to dynamically determine the overall risk score associated with the extracted claim data comprise:
program instructions to identify a plurality of factors associated with collateral information associated with the extracted claim data;
program instructions to assign a weighted value to each identified factor within the identified plurality of factors based on the collateral information associated with the extracted claim data; and
program instructions to calculate an overall risk score by summing the assigned weight values for each identified factor based on the collateral information associated with the extracted claim data.

15. A computer system comprising:
one or more computer processors;
one or more computer readable storage media; and
program instructions stored on the one or more computer readable storage media for execution by at least one of the one or more processors, the program instructions comprising:
program instructions to identify claim data from a received data set associated with a user;
program instructions to analyze the identified claim data based on a historical database of claim information associated with the user;
program instructions to retrieve expert information associated with the analysis of the identified claim data;
program instructions to extract the identified claim data based on a comparison of the analysis of identified claim data and the retrieved expert information associated with the claim data by normalizing a range of features of the analyzed data;
program instructions to dynamically determine an overall risk score associated with the extracted claim data; and
in response to the determined overall risk score meeting or exceeding a predetermined threshold of risk, program instructions to dynamically transmit the extracted claim data to a different computing device associated with a different user.

16. The computer system of claim 15, wherein the program instructions to analyze the identified claim data based on the historical database of claim information comprise:
program instructions to determine the claim data within the identified data has a quantitative impact on an overall risk score of the identified claim data;
program instructions to identify a plurality of eligibility factors within the identified claim data based on the historical database of information associated with the user using a machine learning algorithm and an artificial intelligence algorithm; and
program instructions to compare the determined claim data to the identified plurality of eligibility factors based on the historical database of claim information associated with the user.

17. The computer system of claim 15, wherein the program instructions to retrieve expert information comprise:
program instructions to perform a query for expert information associated with the analyzed claim data;
program instructions to match at least two eligibility factors within a plurality of eligibility factors associated with each expert information within the query of expert opinions based on compliance requirements; and
program instructions to retrieve the expert information with at least two matching eligibility factors within the plurality of eligibility factors.

18. The computer system of claim 17, wherein the program instructions to match the at least two eligibility factors comprise program instructions to link commonalities within the plurality of eligibility factors based on a predetermined set of pre-processing conditions associated with the analyzed claim data.

19. The computer system of claim 15, wherein the program instructions to extract the identified claim data comprise program instructions to import the identified claim data into an intermediate database prior to the normalizing of the received data.

20. The computer system of claim 19, wherein the program instructions stored on the one or more computer readable storage media further comprise:
program instructions to add metadata to the extracted claim data during the importation of the identified claim data into the intermediate database, wherein the metadata is collateral information associated with the user.
